(19) United States
(12) Patent Application Publication
(10) Pub. No.: US 2014/0243780 A1
(43) Pub. Date: Aug. 28, 2014

(54) SYSTEMS AND METHODS FOR REDUCING MUCIN HYPERSECRETION

(71) Applicant: EMPIRE TECHNOLOGY DEVELOPMENT, Wilmington, DE (US)

(72) Inventors: Boris Leschinsky, Mahwah, NJ (US); Jonathan Williams, Montville, NJ (US)

(73) Assignee: EMPIRE TECHNOLOGY DEVELOPMENT, Wilmington, DE (US)

(21) Appl. No.: 13/982,806

(22) PCT Filed: Feb. 28, 2013
(86) PCT No.: PCT/US13/28267

§ 371 Date: Jul. 31, 2013

(51) Int. Cl. A61B 18/14

(52) U.S. Cl. CPC A61B 18/1492; A61B 2018/00315
USPC 604/500; 606/41; 606/42; 604/93.01; 606/34; 606/27

(57) ABSTRACT

Device methods and systems for ablating a mucosal surface to treat patients with mucin hypersecretion is disclosed. An ablation device having a balloon membrane with a plurality of electrodes arranged on an external surface thereof is disclosed. The ablation device may be configured to ablate epithelial tissue of one or more target structures, such as the inner wall of a gallbladder. Each of the plurality of electrodes may be electrically coupled to a controller configured to selectively activate one or more of the plurality of electrodes at a time. The controller may activate less than all of the plurality of electrodes, thereby implementing a partial ablation procedure.

### BACKGROUND

Gallstones are present in a large percentage of the population and can cause inflammation of the gallbladder, infection, severe pain, fever, and in some cases, gallbladder cancer. There are two types of gallstones: cholesterol and pigment, the most common being cholesterol gallstones which occur in 75% of patients having gallstones. The formation of gallstones may be influenced by various factors, such as genetics, age, gender, and certain metabolic factors.

Gallstone treatments include surgical procedures and non-surgical treatments that use drugs or other chemicals to dissolve the gallstones. Such treatments may remove the gallstones, however, they are invasive and have created many potential health complications for patients. Other treatments, such as shock wave lithotripsy, are of limited effectiveness and may be used only for specific types of gallstones. As such, conventional gallstone treatment methods do not provide an effective and simple means for managing gallstones without the need for chronic use of drugs or surgery.

### SUMMARY

The inventions described in this document are not limited to the particular systems, methodologies or protocols described, as these may vary. The terminology used herein is for the purpose of describing particular embodiments only, and is not intended to limit the scope of the present disclosure.

It must be noted that as used herein and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art. As used herein, the term “comprising” means “including, but not limited to.”

Presently disclosed is a method of treating a patient who has gallbladder disease, including for example, mucin hypersecretion. The method comprises ablating either partially or fully a gallbladder mucosa, thereby reducing the rate and chance of gallstone formation.

In an embodiment, a device for ablating a mucosal surface comprises an ablation mechanism, and a controller for controlling the ablation mechanism. In an additional embodiment, an ablation device comprises a catheter having a balloon connected at a distal end of the catheter, wherein the balloon has an internal surface and an external surface, and wherein the external surface comprises an ablation component.

In a further embodiment, a device for partially ablating a mucosal surface, the device comprises an ablation balloon comprising a plurality of electrodes arranged on an external surface thereof; an ablation controller communicatively coupled to the plurality of electrodes, wherein the ablation controller is configured to selectively activate less than all of the plurality of electrodes. Such a device is advantageous for targeting ablation to specific areas of the gallbladder mucosa.

In an embodiment, a device comprises an ablation balloon comprising a plurality of electrodes arranged on an external surface thereof, at least one input device, at least one display device, at least one processor operatively coupled to the ablation balloon; the at least one input device, and the at least one display device; and at least one non-transitory computer-readable storage medium operatively coupled to the at least one processor. The computer-readable storage medium comprises one or more programming instructions that, when executed, causes the at least one processor to receive ablation information from at least one of the plurality of electrodes, present the ablation information on the at least one display device, receive an input from the at least one input device to control operation of the ablation balloon, and selectively activate less than all of the plurality of electrodes responsive to the input received from the at least one input device.

### BRIEF DESCRIPTION OF THE FIGURES

FIG. 1A depicts an illustrative ablation device according to some embodiments.

FIGS. 1B and 1C depict illustrative electrodes according to some embodiments.

FIG. 2 depicts an illustrative ablation device control system according to embodiments.

FIG. 3 depicts a block diagram of illustrative internal hardware that may be used to contain or implement program instructions according to an embodiment.

FIG. 4 depicts an illustration of an ablation device deployed in a gallbladder.

### DETAILED DESCRIPTION

Gallstone disease (cholelithiasis) is a multi-factorial disease. Gallstone detection can be difficult since pigment stones are radiopaque due to their calcium content, and cholesterol stones are radiolucent. Radiopaque objects prevent the passage of radiant energy, such as x-rays, causing the objects to appear dark on exposed film, while radiolucent objects appear lighter because radiant energy passes through the object. Since cholesterol gallstones may not appear on x-rays due to their radiolucency, ultrasound is the preferred method of gallstone detection.

The major components of almost all types of gallstones are free unesterified cholesterol, unconjugated bilirubin, bilirubin calcium salts, fatty acids, calcium carbonates and phosphates, and mucin glycoproteins. Cholesterol gallstones are formed in the gallbladder due to impaired relationships between the major bile components: cholesterol, phospholipids, and bile acids. A critical step in the formation of cholesterol gallstones is nucleation (i.e., the formation of cholesterol monohydrate crystals from supersaturated bile). The rate of nucleation of cholesterol depends upon a critical balance between pro- and anti-nucleating factors in bile. Studies have shown that bile from gallstone patients displays more rapid cholesterol crystallization than bile from non-diseased subjects. Mucin, a high molecular weight glycoprotein secreted by the gallbladder mucosa epithelium, is a pronucleating agent in experimental and human gallstone disease. Mucin hypersecretion is a known factor that leads to an imbalance between antinucleating and pronucleating factors in bile and causes excessive crystallization of cholesterol. In most cases, the stones are benign and patients are asymptomatic. Current gallstone treatments include surgical gallbladder removal (cholecystectomies), litholytic treatment, shock wave lithotripsy, and combined shock wave lithotripsy and litholytic treatment.

Gallstone surgery is one of the most common intestinal surgeries. Laparoscopic cholecystectomy is currently considered the gold standard for treatment. Although considered safe and effective, it is still associated with certain levels of complications such as infection at the incision point, internal bleeding, risk of general anesthesia, injury to the common bile duct, injury to the small intestine, and bile leaks in the abdominal cavity. Retention of the gallbladder and its function is advantageous so as to avoid complications and have a faster recovery. There is a need for an effective and simple means for prevention of gallstones without the need for chronic use of drugs or surgery.

Ablation, as described herein, is a method of removing tissue non-surgically from the body where it may cause cell death, osmotic lysis, apoptosis, necrosis, or mitotic arrest. A disclosed method of treating a patient having mucin hypersecretion comprises partially ablating a gallbladder mucosa. In such an example, the ablation reduces the surface area and number of secretory cells producing mucin. Partial ablation comprises ablating less than 100% of a luminal mucosa of the gallbladder. Such methods reduce the secretion of mucin, thereby reducing or preventing future stone formation. In certain embodiments, a portion of the inner wall mucosa is ablated which reduces the number of epithelial cells producing mucin and releasing mucin into the gallbladder. In instances where secretory stem cells are targeted, such a reduction can be permanent. In some embodiments, the portion of a luminal mucosa of the gallbladder that is ablated may be about 10% of the luminal mucosa, about 20% of the luminal mucosa, about 30% of the luminal mucosa, about 40% of the luminal mucosa, about 50% of the luminal mucosa, about 60% of the luminal mucosa, about 70% of the luminal mucosa, about 80% of the luminal mucosa, about 90% of the luminal mucosa, or any percentage between any two of the listed values.

Desired reduction of mucin production can be achieved by ablation of a specific surface area of the gallbladder wall. In some embodiments, the ablation may be targeted to the fundus mucosa of the gallbladder. In other embodiments, the ablation may be targeted to the corpus mucosa of the gallbladder. In other embodiments, the ablation may be targeted to the infundibulum mucosa of the gallbladder. In further embodiments, the ablation may be targeted to a part of the fundus mucosa, a part of the corpus mucosa, a part of the infundibulum mucosa, and/or combinations thereof. Once the hypersecretion of mucin is halted, the balance of pronucleating and antinucleating factors in bile can be restored to normal levels and further crystallization of cholesterol is inhibited, thereby leading to reduced propensity for further stone formation.

The terminology used in the description is for the purpose of describing the particular versions or embodiments only, and is not intended to limit the scope. In one aspect, the present disclosure is directed toward an ablation device configured to ablate mucosal tissue using electrodes. An ablation device as described herein is any device that uses an ablation mechanism with or without a controller which controls the device. Such mechanisms may be used to partially or fully ablate a surface depending on the needs of those of average skill in the art. In another aspect, the present disclosure is directed towards a device for partially ablating a mucosal surface using an ablation mechanism and a controller that controls the ablation mechanism. The ablation mechanism may comprise one or more of a chemical component, an electrical component, a mechanical component, and a thermal component. The aspect directed towards a device for partially ablating a mucosal surface using an ablation mechanism and a controller to control the ablation mechanism may comprise, but is not limited to, an infrared ablation device, a cryoablation device, a thermal ablation device, a radiofrequency ablation device, a gamma radiation ablation device, an electrocautery ablation device, or any combination thereof.

For example, an infrared ablation device may be any device that uses infrared light for ablation including, but not limited to, devices that use lasers to ablate surfaces. For example, a cryoablation device may be any device that uses freezing for ablation including, but not limited to, devices using liquid nitrogen and devices that use pressurized gas to ablate surfaces. For example, a thermal device may be any device that uses heat for ablation including, but not limited to, devices using heat probes to apply direct heat application to ablate surfaces. For example, a radiofrequency device may be any device that uses electrical conduction for ablation including, but not limited to, devices that deliver heat generated from high frequency alternating current through energy-emitting probes to ablate tissue. For example, a gamma radiation device may be any device that uses radiosurgery for ablation including, but not limited to, devices using a gamma knife to deliver ionizing radiation to ablate surfaces. For example, an electrocautery device may be any device that uses an electrical circuit for ablation including, but not limited to, devices using a probe with a tip that contains two electrodes, which enable completion of an electrical circuit at the end to ablate surfaces. A chemical component used in an ablation mechanism may be in a gas phase or a liquid phase. The chemical component of the ablation mechanism may include, without limitation, acetic acid solution, ethanol, and/or silver nitrate.

According to an aspect directed toward an ablation device using electrodes, the ablation device may comprise an ablation balloon having a plurality of electrodes arranged on or within an external surface of the ablation balloon. The ablation balloon may be inserted into an internal organ, such as the gallbladder, esophagus, bladder, or uterus, and may be inflated such that the external surface contacts the inner wall mucosa of the organ. When the ablation balloon is inflated such that it contacts the inner wall mucosa, at least a portion of the plurality of electrodes is in contact with at least a portion of the inner wall mucosa. The ablation device may be controlled by a controller configured to energize the plurality of electrodes. In an embodiment, energized electrodes may emit radio frequency (RF) energy that operates to ablate tissue sufficiently exposed thereto. The plurality of electrodes may be individually controlled by the controller such that less than all of the plurality of electrodes may be activated, thereby resulting in partial ablation of the organ or structure.

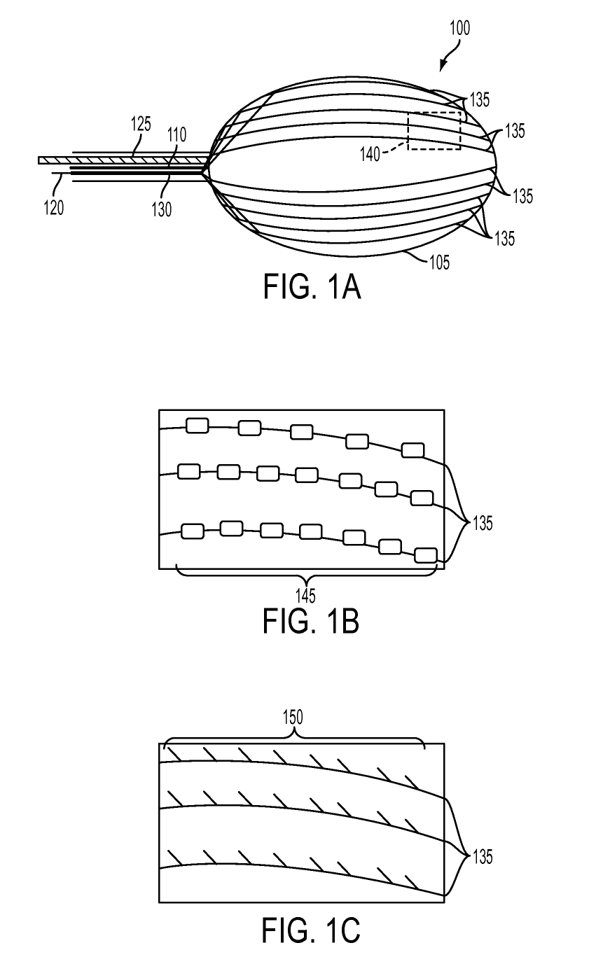

FIG. 1A depicts an illustrative ablation device according to some embodiments. As shown in FIG. 1A, an ablation device 100 may comprise a balloon membrane 105 configured to be inflated by an inflation/deflation lumen 125. According to some embodiments, the balloon membrane 105 may be made of a biocompatible polymer, such as polyurethane. The inflation/deflation lumen 125 may inflate the balloon membrane 105 through various methods. For example, the inflation/deflation lumen 125 may be used to force air into the interior of the balloon membrane 105 that pressurizes the interior of the balloon membrane, causing it to expand. In another example, the inflation/deflation lumen 125 may be used to fill the interior of the balloon membrane 105 with one or more fluids that cause the balloon membrane to expand.

Although the balloon membrane 105 depicted in FIG. 1A has a substantially round or oval shape, embodiments are not so limited. The balloon membrane 105 may be configured to have any shape capable of operating according to embodiments described herein, including, without limitation, circular, pear, peanut, and shapes substantially conforming therewith.

The balloon membrane 105 depicted in FIG. 1A is illustrated as being at least partially inflated. For deployment within a human body, the ablation balloon 105 and certain other components of the ablation device 100 may be collapsed to a size and/or shape capable of entry into one or more orifices. For example, the ablation device 100 may be introduced into the gastrointestinal tract through the mouth or into the gallbladder through the opening of the sphincter of Oddi using an endoscope. In one embodiment, the ablation balloon 105 may be connected to a catheter. A guide wire lumen 110 attached to or configured to receive a guide wire 120 may be disposed within or connected to the ablation balloon 105. The guide wire 120 may be used to push or otherwise guide the ablation balloon 105, along with the elements of the ablation device 100 contained therein, into the human body to the intended target structure. The target structure may comprise an internal organ, tissue, or other collection of cells, such as a gallbladder. Embodiments provide that the guide wire lumen 110 may be configured to receive guide wires of varying gauges, such as about 0.01 inches (“10 gauge”), about 0.02 inches (“20 gauge”), about 0.03 inches (“30 gauge”), about 0.04 inches (“40 gauge”), about 0.05 inches (“50 gauge”), and ranges between any two of these values (including endpoints).

A plurality of electrodes 135 may be arranged on or within the external surface of the ablation balloon 105 and isolated from the internal volume of the ablation balloon. The ablation device 100 may comprise any number of electrodes, including about 5 electrodes, about 10 electrodes, about 20 electrodes, about 30 electrodes, about 50 electrodes, or any range between two of these numbers (including endpoints). According to some embodiments, each of the plurality of electrodes 135 may comprise an array of electrodes. A detailed view, designated by area 140, of the plurality of electrodes 135 configured according to some embodiments is depicted in FIGS. 1B and 1C. When activated, each of the plurality of electrodes 135 may emit energy sufficient to ablate tissue. In an embodiment, the energy comprises radio frequency (RF) signals. In such an embodiment, the plurality of electrodes 135 may be connected to an RF energy source configured to provide various levels of RF energy. For example, the RF energy source may provide RF energy at a frequency of about 100 kilohertz (kHz), about 200 kHz, about 300 kHz, about 500 kHz, about 1000 kHz, or a range between any two of these values (including endpoints).

In some embodiments, the depth of ablation is influenced by the choice of RF frequency. In some embodiments, the surface cells (mucosal cells) are the ablation target, and the RF frequency, energy and durations levels are optimized for this effect.

An electrical conduit lumen 130 may be electrically coupled to at least a portion of the plurality of electrodes 135. The electrical conduit lumen 130 may have one or more circuits, electrical leads, electrodes, or other such elements (e.g., “electrical elements”) that are configured to establish an electrical connection with the plurality of electrodes 135. In one embodiment, the external surface of the electrical conduit lumen 130 may contact the inner surface of the ablation balloon 105 such that at least a portion of the electrical elements establish an electrical connection with at least a portion of the plurality of electrodes 135. In another embodiment, each of the plurality of electrodes 135 may have a lead that connects to the electrical conduit lumen 130.

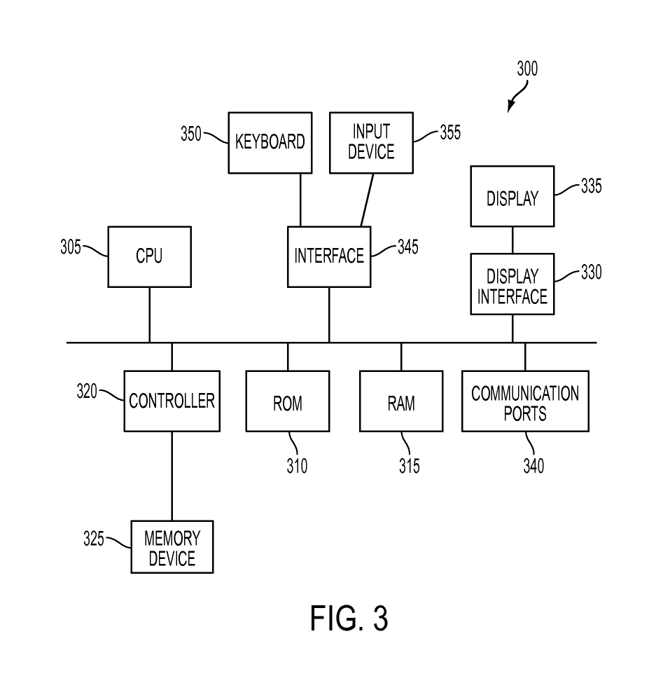

The electrical conduit lumen 130 may be connected to a controller (not shown; depicted in FIG. 3 and described in reference thereto) configured to control the activation of each of the plurality of electrodes 135. The electrical conduit lumen 130 may comprise at least one electrical lead 120 that is connected to the controller. The electrical lead 120 may comprise a bundle of leads that provide a separate path for electrical signals for each of the plurality of electrodes 135. According to some embodiments, the electrical lead 120 may provide for two-way signal transmission between the electrical conduit lumen 130 and the controller. In this manner, the controller may send control signals to the electrical conduit lumen 130, for example, to control activation of the plurality of electrodes 135. The electrical conduit lumen 130 may also send signals to the controller, for instance, comprising information associated with the plurality of electrodes 135, such as voltage resulting from current emitted by the electrodes.

The ablation device 100 is configured such that less than all of the plurality of electrodes 135 may be activated at any time. For example, one electrode, two electrodes, about 2% of the plurality of electrodes, about 5% of the plurality of electrodes, about 10% of the plurality of electrodes, about 25% of the plurality of electrodes, about 33% of the plurality of electrodes, about 50% of the plurality of electrodes, about 75% of the plurality of electrodes, about 100% of the plurality of electrodes, or ranges between any two of these values (including endpoints) may be activated. In this manner, the ablation device 100 may effect a partial ablation procedure, as described according to embodiments provided herein. The number and the location of activated electrodes may be controlled by the controller.

FIGS. 1B and 1C depict a detailed view of electrodes of the ablation device according to some embodiments. As described above, each of the plurality of electrodes 135 may be configured as an array of electrodes 145, 150. Embodiments provide that the array of electrodes 145, 150 may comprise clusters of electrodes of various numbers, including about 5 electrodes, about 10 electrodes, about 20 electrodes, about 30 electrodes, about 50 electrodes, or ranges between any two of these numbers (including endpoints). As shown in FIG. 1B, the array of electrodes 145 may comprise electrical elements arranged on the external surface of the ablation balloon 105. In FIG. 1C, the array of electrodes 150 may comprise electrical elements that protrude from the surface of the ablation balloon 105 and, for example, pierce tissue in contact with the ablation balloon. Electrode arrays configured according to embodiments described herein are not limited to the exact electrical elements depicted in FIG. 1B or 1C, as these are provided as illustrative and non-restrictive embodiments.

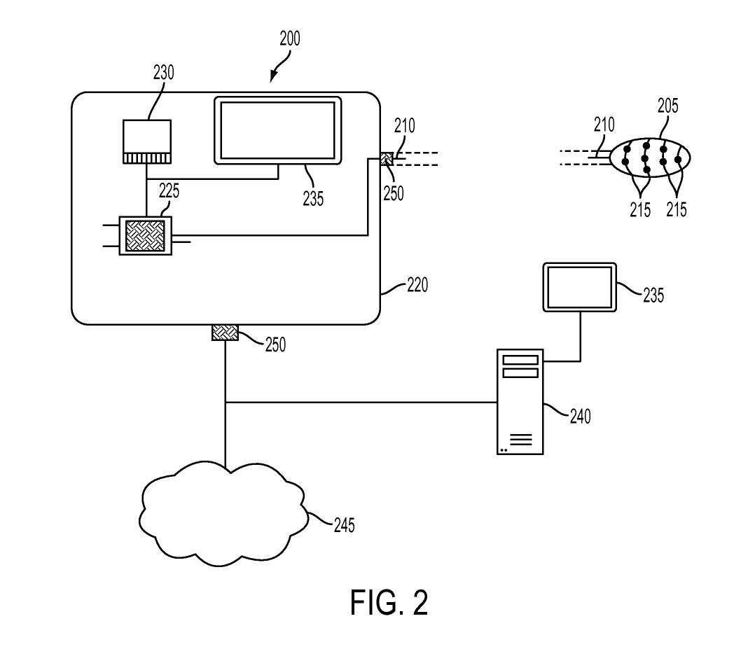

FIG. 2 depicts an illustrative ablation device controller system according to some embodiments. The controller system 200 may generally comprise a processor 225, a non-transitory memory 230 or other storage device for housing programming instructions, data or information regarding one or more applications, and other hardware, including, for example, the central processing unit (CPU) 305, read only memory (ROM) 310, random access memory 315, communication ports 340, controller 320, and/or memory device 325 depicted in FIG. 3 and described below in reference thereto. The processor 225 may execute one or more software programs, such as an ablation device control application, for operating an ablation device 250 or particular aspects thereof.

The components of the controller system 200 may be housed within a case 220 having one or more communication ports 250. At least one of the communication ports 250 may be configured to link the controller system 200 with an electrical lead 210 of the ablation device 205. The electrical lead 210 may be electrically coupled with an electrical conduit lumen (not shown) (e.g., electrical conduit lumen 130 of FIG. 1A) that is electrically coupled with the arrays of electrodes 215 of the ablation device 205. The processor 225 may be connected to the electrical lead 210 such that the processor may receive electrical signals from and send electrical signals to the each of the arrays of electrodes 215.

At least one communications port 250 may provide a connection to a computing device 240 and/or a network 245. The computing device may comprise various types of computing devices, including, without limitation, a server, personal computer (PC), tablet computer, computing appliance, or smart phone device. Non-restrictive examples of networks 245 include communications networks or health information networks (e.g., picture archiving and communications system (PACS)). The communications ports 250 may provide a connection to the computing device or networks through communication protocols known to those having ordinary skill in the art, such as Ethernet and Wi-Fi. In this manner, information associated with and control of the ablation device 205 may be accessible by systems outside of the actual ablation control system 200 unit contained within the case 220.

The ablation device control application executed by the processor 225 may be configured to present an ablation device user interface on, for example, a display device 235. The ablation device user interface may provide users with various control functions and information associated with the ablation device and operation thereof. From the ablation device user interface, users may control inflation/deflation of the ablation device 205, selectively activate one or more of the arrays of electrodes 215, and perform other functions related to ablating tissue using the ablation device, such as monitoring the ablation process or determining the number of electrodes contacting the wall of the target organ or structure. According to some embodiments, the ablation device user interface may also be presented through a display device 235 communicatively coupled with a computing device 240 or otherwise available over the network 245.

In an embodiment, the ablation device user interface may provide a function that controls one or more of the arrays of electrodes 215 to emit an interrogating current. For example, the arrays of electrodes 215 may emit an alternating current (AC) of about 10 microamperes (μA), about 20 μA, about 30 μA, about 40 μA, about 50 μA, about 60 μA, about 70 μA, about 80 μA, about 90 μA, about 100 μA, and ranges between any two of these values (including endpoints). The processor 225 may receive signals pertaining to the voltage resulting from the interrogating current. The ablation device control application may be configured to present the voltage and/or current information on the ablation device user interface and/or analyze the voltage information to determine how many of the electrodes have reached the wall of the target structure (e.g., gallbladder).

The ablation device control application may be configured according to some embodiments to provide a function through the ablation device user interface that allows a user to control one or more of the arrays of electrodes 215 to emit ablation-level RF. The emission of ablation-level RF may be initiated to ablate the wall of a target structure during an ablation procedure. The processor 225 may receive electrical information resulting from the emission of the ablation-level RF that is associated with the effectiveness of the ablation procedure. For example, the changing electrical signature of the bio-impedance of the wall of the target structure and/or delivered RF energy may provide an indication of how the ablation procedure is progressing. In some embodiments the controller system 200, through the processor 225, may monitor instantaneous RF power (for example, RF current and voltage), monitor RF current, or combinations thereof and use it for control of the process.

In an embodiment, the monitoring process uses measurements of bio-impedance of the wall of the target structure to determine when to terminate the application of energy to the tissue. Bio-impedance may be measured by various methods, for example, directly via the use of low level interrogating currents and/or by the change in RF ablation current or voltage. In an embodiment where bio-impedance is measured via the use of interrogating currents, the currents are applied on a periodic basis, for instance, inter-leaved with application of the ablation-level RF. In some embodiments, an early decrease of impedance indicates adequate tissue ablation. In some embodiments, an early decrease of impedance from about 1 to 20 ohms indicates adequate tissue ablation. In some embodiments, an early decrease of impedance from about 2 to 20 ohms indicates adequate tissue ablation. In some embodiments, an early decrease of impedance from about 5 to 20 ohms indicates adequate tissue ablation. In some embodiments, an early decrease of impedance from about 5 ohms or greater indicates adequate tissue ablation. In other embodiments, an early decrease of impedance from about 10 ohms or greater indicates adequate tissue ablation. In the above mentioned embodiments, these changes in impedance may indicate that sufficient ablation-level RF energy has been applied to ablate the target tissue. If tissue impedance greatly increases during ablation, the local tissue temperature may have reached 100° C. or higher and resulted in desiccation or charring of the tissue due to overheating of the tissue. In some embodiments, if the impedance value increases greater than a predetermined rate, an alarm may sound from the controller to indicate the overheating of the tissue.

In an embodiment, the ablation device user interface may display the electrical information resulting from the emission of the ablation-level RF (e.g., bio-impedance). In another embodiment, the ablation device control application may be configured to analyze the electrical information resulting from the emission of the ablation-level RF to generate an output pertaining to the progress of the ablation procedure. For instance, the ablation device control application may analyze the bio-impedance information to generate a message pertaining to the ablation procedure, such as a warning for values out of range or a message that ablation in a particular area is complete.

According to some embodiments, the ablation control user interface may provide functions for activating less than all of the arrays of electrodes 215. For example, the ablation control user interface may provide an input function that accepts a value pertaining to the percent or number of electrodes to activate. In another example, the ablation control user interface may provide a function that allows a user to select specific electrodes, electrodes at specific regions of the ablation device 250, and/or electrodes associated with a range of bio-impedance values for activation. The processor 225 may be configured to transmit signals to the ablation device 250 that control activation of electrodes as selected through the ablation control user interface.

FIG. 3 depicts a block diagram of exemplary internal hardware that may be used to contain or implement program instructions described herein, such as the ablation device control application or components thereof. A bus 300 serves as the main information highway interconnecting the other illustrated components of the hardware. CPU 305 is the central processing unit of the system, performing calculations and logic operations required to execute a program. CPU 305, alone or in conjunction with one or more of the other elements disclosed in FIG. 2, is an exemplary processing device, computing device or processor as such terms are used in this disclosure. Read only memory (ROM) 310 and random access memory (RAM) 315 constitute exemplary memory devices.

A controller 320 interfaces with one or more optional memory devices 325 to the system bus 300. These memory devices 325 may include, for example, an external or internal DVD drive, a CD ROM drive, a hard drive, flash memory, a USB drive, or the like. As indicated previously, these various drives and controllers are optional devices.

Program instructions, software or interactive modules for providing the digital marketplace and performing analysis on any received feedback may be stored in the ROM 310 and/or the RAM 315. Optionally, the program instructions may be stored on a tangible computer readable medium such as a compact disk, a digital disk, flash memory, a memory card, a USB drive, an optical disc storage medium, such as a Blu-ray™ disc, and/or other recording medium.

An optional display interface 330 may permit information from the bus 300 to be displayed on the display 335 in audio, visual, graphic or alphanumeric format. Communication with external devices may occur using various communication ports 340. An exemplary communication port 340 may be attached to a communications network, such as the Internet, or an intranet. Other exemplary communication ports 340 may comprise a serial port, a RS-232 port, and a RS-485 port.

The hardware may also include an interface 345 which allows for receipt of data from input devices such as a keyboard 350 or other input device 355 such as a mouse, a joystick, a touch screen, a remote control, a pointing device, a video input device, and/or an audio input device.

The ablation method may provide a decrease in mucin production resulting in about 10% reduction, about 20% reduction, about 30% reduction, about 40% reduction, about 50% reduction, about 60% reduction, about 70% reduction, about 80% reduction, about 90% reduction, or any percentage between any of these listed values. In some embodiments, the method comprises removing at least one gallstone. It may be necessary to remove several gallstones. In some embodiments, the gallbladder may necessitate irrigation prior to the ablation method.

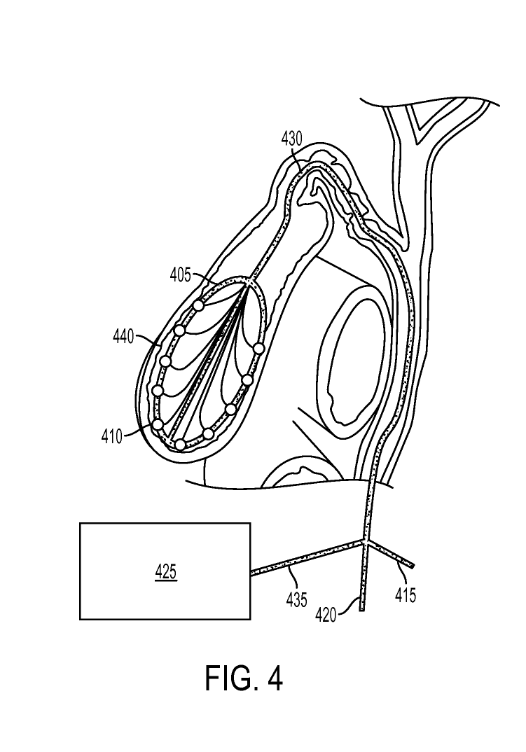

FIG. 4 depicts an illustrative ablation device according to some embodiments comprised of a balloon catheter 430 having an ablation balloon 405 connected at a distal end of the balloon catheter inserted into a targeted structure, such as a gallbladder, and partially inflated to contact the luminal mucosa 440. The ablation controller 425 is communicatively coupled to the plurality of electrodes 410 by the electrical conduit lumen 435 according to some embodiments. The external surface of the ablation balloon 405 may be comprised of a balloon inflation lumen 415 which covers a guidewire lumen 420 that may aid in the insertion of the ablation device into the patient.

### EXAMPLES

### Example 1

### Ablation Device

An ablation device will be manufactured for ablating mucosal tissue of the inner wall of a gallbladder. The ablation device will include a balloon membrane made out of polyurethane and twenty arrays of electrodes equally-spaced and circumferentially orientated about the external surface of the balloon membrane. Each array of electrodes will include thirty gold-plated copper electrodes. An electrical conduit lumen will be electrically coupled to each of the arrays of electrodes. The electrical conduit lumen will have an electrical lead configured to provide an electrical connection to each of the arrays of electrodes to an ablation device control system. The electrical conduit lumen will also be connected to an RF energy source capable of providing about 400 kHz of RF energy.

The ablation device control system will execute ablation device control software that presents a user interface on a touch screen display device. The user interface will include virtual buttons to inflate the ablation device, deflate the ablation device, select a number of electrodes to activate, and select a level of energy for activated electrodes.

### Example 2

### Method of Reducing Hypersecretion in the Gallbladder

A patient diagnosed with mucin hypersecretion in the gallbladder will receive ablation surgery to reduce mucin hypersecretion. The patient will undergo the procedure to achieve 20-30% reduction of mucin production by ablation of a corresponding surface area of the gallbladder luminal mucosa. Prior to the partial ablation procedure, the balloon of an example ablation device will be collapsed and tightly wrapped to prepare for deployment into the patient. It will be introduced into the gallbladder via a guide wire (outer diameter of 0.01 inches) using an endoscope placed at the opening of the sphincter of Oddi. A pear-shaped balloon will then be inflated using saline until it contacts the gallbladder luminal mucosa. The patient will undergo flushing of the gallbladder prior to balloon inflation if necessary. In order to detect proper positioning and inflation of the balloon, bio-impedance feedback from the gallbladder luminal mucosa will be measured with an interrogating AC current emitted from the desired electrodes. Ultrasound imaging will be used to determine the gallbladder size and luminal mucosa surface area necessary to achieve 20-30% reduction of mucin production. Two arrays of electrodes, consisting of 10 electrodes in each array, will be activated to cause 20-30% reduction of mucin production as determined from the ultrasound.

The electrodes will be activated, and then the electrodes will emit ablation-level RF energy to the gallbladder luminal mucosa for partial ablation. During the ablation process, the instantaneous power delivered to the wall tissue will be continuously monitored to assure efficacy and patient safety. The partial ablation will be verified by observing the changing electrical signature of the luminal mucosa bio-impedance. Once the partial ablation is complete, the saline will be removed to deflate the balloon, and the device will then be removed from the patient.

This disclosure is not limited to the particular systems, devices and methods described, as these may vary. The terminology used in the description is for the purpose of describing the particular versions or embodiments only, and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases at least one and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases one or more or at least one and indefinite articles such as “a” or an (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C′” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C′” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of treating a patient having mucin hypersecretion, comprising:
partially ablating a gallbladder mucosa.

2. The method of claim 1, wherein the step of partially ablating further comprises ablating less than 100% of a luminal mucosa of the gallbladder.

3. The method of claim 1, wherein the step of partially ablating further comprises ablating a portion of a luminal mucosa of the gallbladder, wherein the portion is selected from the group consisting of about 10% of the luminal mucosa, about 20% of the luminal mucosa, about 30% of the luminal mucosa, about 40% of the luminal mucosa, about 50% of the luminal mucosa, about 60% of the luminal mucosa, about 70% of the luminal mucosa, about 80% of the luminal mucosa, and about 90% of the luminal mucosa.

4. The method of claim 1, further comprising:
removing at least one gallstone.

5. The method of claim 1, further comprising:
irrigating the gallbladder.

6. A device for partially ablating a mucosal surface, the device comprising:
an ablation mechanism; and
a controller for controlling the ablation mechanism.

7. The device of claim 6, wherein the ablation mechanism comprises one or more of a chemical component, an electrical component, a mechanical component, or a thermal component.

8. The device of claim 6, wherein the ablation mechanism comprises an infrared ablation device, a cryoablation device, a thermal ablation device, a radio frequency ablation device, a gamma radiation ablation device, or an electrocautery ablation device.

9. The device of claim 6, wherein the ablation mechanism comprises a chemical component selected from the group consisting of acetic acid solution, ethanol, and silver nitrate.

10. (canceled)

11. An ablation device comprising:
a catheter having a balloon connected at a distal end of the catheter, wherein the balloon has an internal surface and an external surface, and wherein the external surface comprises an ablation component.

12. The device of claim 11, wherein the external surface of the balloon is configured to contact the luminal mucosa.

13. The device of claim 11, wherein the ablation component comprises a thermal component, wherein the thermal component is configured to heat a chemical within the balloon catheter.

14. The device of claim 11, wherein the ablation component comprises a radio frequency ablation component.

15. The device of claim 14, wherein the radio frequency ablation component comprises at least one radio frequency electrode located on the external surface of the balloon.

16. The device of claim 14, wherein the radio frequency ablation component comprises a plurality of electrodes located in an array on the external surface of the balloon.

17. (canceled)

18. The device of claim 14, wherein the ablation component further comprises an ionizing gas flow.

19. A device for partially ablating a mucosal surface, the device comprising:
an ablation balloon comprising a plurality of electrodes arranged on an external surface thereof; and
an ablation controller communicatively coupled to the plurality of electrodes, wherein the ablation controller is configured to selectively activate less than all of the plurality of electrodes.

20. (canceled)

21. (canceled)

22. The device of claim 19, wherein the ablation controller is configured to receive ablation information indicating whether at least one of the plurality of electrodes is contacting a surface of a target structure.

23. The device of claim 19, wherein the ablation controller is configured to receive, from at least one of the plurality of electrodes, ablation information comprising bio-impedance information.

24. The device of claim 23, wherein the ablation controller is configured to verify an ablation procedure based on the bio-impedance information.

25. The device of claim 23, wherein bio-impedance information comprises a changing electrical signature of the bio-impedance information.

26. The device of claim 19, wherein the ablation controller is configured to receive ablation information from at least one of the plurality of electrodes, wherein the ablation controller is configured to activate at least one of the plurality of electrodes to emit an interrogating current.

27. (canceled)

28. The device of claim 26, wherein the ablation controller is configured to monitor voltage resulting from the interrogating current to indicate whether at least one of the plurality of electrodes is contacting a surface of a target structure.

29. The device of claim 19, wherein less than all of the plurality of electrodes comprises one of about 25% of the plurality of electrodes, about 50% of the plurality of electrodes, and about 75% of the plurality of electrodes

30. (canceled)

31. (canceled)

32. (canceled)

33. (canceled)

34. (canceled)

35. An ablation system comprising:
an ablation balloon comprising a plurality of electrodes arranged on an external surface thereof;
at least one input device;
at least one display device;
at least one processor operatively coupled to the plurality of electrodes, the at least one input device, and the at least one display device; and
at least one non-transitory computer-readable storage medium operatively coupled to the at least one processor, the at least one non-transitory computer-readable storage medium comprising one or more programming instructions that, when executed, cause the at least one processor to:
receive ablation information from at least one of the plurality of electrodes;
present the ablation information on the at least one display device; and
receive input from the at least one input device to control at least one operation of the ablation balloon, wherein the at least one operation of the ablation balloon comprises selectively activating less than all of the plurality of electrodes.

36. The system of claim 35, wherein the at least one operation of the ablation balloon further comprises inflation of the ablation balloon.

37. The system of claim 35, wherein less than all of the plurality of electrodes comprises one of about 25% of the plurality of electrodes, about 50% of the plurality of electrodes, and about 75% of the plurality of electrodes.

38. (canceled)

39. (canceled)

40. The system of claim 35, wherein the one or more programming instructions, when executed, further cause the at least one processor to receive ablation information from the plurality of electrodes.

41. The system of claim 40, wherein the one or more programming instructions, when executed, further cause the at least one processor to determine a status of the ablation procedure based on the ablation information.

42. The system of claim 41, wherein the ablation information comprises bio-impedance information.

43. (canceled)
